(19) United States
(12) Patent Application Publication
(10) Pub. No.: US 2024/0268947 A1
(43) Pub. Date: Aug. 15, 2024

(54) STENT GRAFT WITH FEATURES FOR SIDE BRANCH VESSEL PERFUSION

(71) Applicant: W. L. Gore & Associates, Inc., Newark, DE (US)

(72) Inventors: Dustin C. Burkart, Bellemont, AZ (US); Kevin K. Coffman, Phoenix, AZ (US); Kimberley N. Wunder, Phoenix, AZ (US)

(21) Appl. No.: 18/436,294

(22) Filed: Feb. 8, 2024

Related U.S. Application Data
(60) Provisional application No. 63/444,382, filed on Feb. 9, 2023.

(51) Int. Cl. A61F 2/07; A61F 2/06; A61F 2/954

(52) U.S. Cl. CPC A61F 2/07; A61F 2/954; A61F 2002/067

(57) ABSTRACT

An endovascular treatment system (e.g., for treating aortic aneurysms) including branch features for perfusing branch vessels, such as the renal, celiac, and mesenteric arteries.

### CROSS-REFERENCE TO RELATED APPLICATION

This application claims the benefit of Provisional Application No. 63/444,382, filed Feb. 9, 2023, which is incorporated herein by reference in its entirety for all purposes.

### BACKGROUND

Aneurysms occur in blood vessels at sites where, due to age, disease or genetic predisposition of the patient, the strength or resilience of the vessel wall is insufficient to prevent ballooning or stretching of the wall as blood passes through. If the aneurysm is left untreated, the blood vessel wall may expand and rupture, often resulting in death.

To prevent rupturing of an aneurysm, a stent graft may be percutaneously introduced into a blood vessel and deployed to span the aneurysmal sac. Stent grafts include a graft fabric secured to a cylindrical scaffolding or framework of one or more stents. The stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

However, various endovascular repair procedures such as the exclusion of an aneurysm require a stent graft to be implanted adjacent to a vascular bifurcation. Often the aneurysm extends into the bifurcation requiring the stent graft to be placed into the bifurcation. A bifurcated stent graft is therefore required in these cases. Modular stent grafts, having a separate main body and branch component are often preferred in these procedures due to the ease and accuracy of deployment. See U.S. Patent Application No. 2008/0114446 to Hartley et al. for an example of a modular stent graft having separate main body and branch stent components. In the Hartley et al. publication, the main body stent has a fenestration in the side wall that is tailored to engage and secure the side branch stent. The side branch stent in such a configuration is in a “line to line” interference fit with the main body fenestration, causing a potential compromise to the fatigue resistance of the stent-to-stent junction. U.S. Pat. No. 6,645,242 to Quinn presents a more robust stent-to-stent joining configuration. In the Quinn patent, a tubular support, internal to the main body stent, is incorporated to enhance the reliability of the stent to stent joining. U.S. Pat. No. 10,653,540 provides further enhancements to these concepts, addressing a highly conformable stent graft with a portal for a side branch device.

### SUMMARY

Various examples relate to an endovascular treatment system including branch features for perfusing branch vessels, such as the renal arteries, superior mesenteric artery, or others.

According to one example, (“Example 1”), an endovascular treatment system includes a main body component having a first end, a second end, and an inner lumen, the main body including a first internal tube and a second internal tube located within the inner lumen, each of the first and second internal tubes having an origin oriented toward the first end of the main body component and an external opening in an outer surface of the main body component; and a plurality of side branch components, including a first side branch component and a second side branch component, each configured to be received in one of the first and second internal tubes and to extend from the external openings in the outer surface of the main body component, wherein the system is configured to be endovascularly implanted in, and to treat the abdominal aorta, the main body component being configured to be anchored in the abdominal aorta at a location that is superior of a location of renal arteries of the patient such that the plurality of side branch components are anchorable in renal arteries of the patient for perfusion thereof, the main body component including at least one of a fenestration feature and a scallop feature configured to align to a superior mesenteric artery of a patient.

According to another example, (“Example 2”), and further to Example 1, the first and second internal tubes are circumferentially offset by approximately 180 degrees.

According to another example, (“Example 3”), and further to Examples 1 or 2, the first and second internal tubes are circumferentially offset by approximately 160 degrees.

According to another example, (“Example 4”), and further to any of Examples 1 to 3, the first and second internal tubes are circumferentially offset by approximately 140 degrees.

According to another example, (“Example 5”), and further to any of Examples 1 to 4, the main body component has a larger, proximal inlet and two smaller, distal outlets defined by two legs.

According to another example, (“Example 6”), and further to Example 5, the two legs include a short leg and a long leg.

According to another example, (“Example 7”), and further to any of Examples 1 to 4, wherein the main body component has a larger, single proximal inlet and a larger, single distal outlet.

According to another example, (“Example 8”), and further to any of Examples 1 to 7, the main body has an outer surface that projects radially outward proximate to the external opening at locations corresponding to an outer profile of each of the internal tubes such that the external openings are longitudinally-oriented.

According to another example, (“Example 9”), and further to any of Examples 1 to 8, the origins of each of the first and second internal tubes are oriented toward the first end of the main body and are longitudinally aligned with the inner lumen of the main body, and further wherein the external openings of each of the first and second internal tubes are longitudinally aligned to the inner lumen.

According to another example, (“Example 10”), and further to any of Examples 1 to 8, the main body has an outer surface and further wherein the external openings of the first and second internal tubes are flush with the outer surface of the main body such that the external openings are radially-oriented.

According to another example, (“Example 11”), and further to any of Examples 1 to 7 or 10, the origins of each of the first and second internal tubes are oriented toward the first end of the main body and are longitudinally aligned with the inner lumen of the main body, and further wherein the external openings of each of the first and second internal tubes extend transverse to the inner lumen.

According to another example, (“Example 12”), and further to any of Examples 1 to 11, the origin of at least one of the first and second internal tubes are inset from the first end of the main body by an inset distance.

According to another example, (“Example 13”), and further to any of Examples 1 to 12, the inset distance is from 5 mm to 10 mm.

According to another example, (“Example 14”), and further to any of Examples 1 to 13, the external opening of at least one of the first and second internal tubes are offset from the first end of the main body by an offset distance of no more than 40 mm.

According to another example, (“Example 15”), a method of delivering an endoprosthesis to a treatment site in vasculature of a patient includes delivering a main body component having a first end, a second end, and an inner lumen to a location in the abdominal aorta such that the first end of the main body is positioned at a location superior to renal arteries of the patient, wherein the main body including a first internal tube and a second internal tube located within the inner lumen, each of the first and second internal tubes having an origin oriented toward the first end of the main body component and an external opening in an outer surface of the main body component. The method also includes aligning at least one of a fenestration feature and a scallop feature of the main body component to a superior mesenteric artery of the patient. And, the method includes delivering a plurality of side branch components, including a first side branch component in the first internal tube and a second side branch component in the second internal tubes such that the first and second side branch components extend from respective ones of the external openings in the outer surface of the main body component and anchoring the first end of the main body component superior to the renal arteries and anchoring the first and second branch components in respective ones of the renal arteries for perfusion thereof.

According to another example, (“Example 16”), and further to Example 15, the first and second internal tubes of the main body component are circumferentially offset by approximately 180 degrees.

According to another example (“Example 17”), and further to Examples 15 or 16, the first and second internal tubes are circumferentially offset by approximately 160 degrees.

According to another example, (“Example 18”), and further to Examples 15 or 16 the first and second internal tubes are circumferentially offset by approximately 140 degrees.

According to another example (“Example 19”), and further to any of examples 15 to 18, the main body component has a larger, proximal inlet and two smaller, distal outlets defined by two legs.

According to another example, (“Example 20”), an endovascular treatment system includes a main body component having a first end, a second end, and an inner lumen, the main body including a first internal tube and a second internal tube located within the inner lumen, each of the first and second internal tubes having an origin oriented toward the first end of the main body component and an external opening in an outer surface of the main body component, the first and second internal tubes being circumferentially offset from approximately 140 degrees to approximately 180 degrees from one another, the main body having an outer surface that projects radially outward proximate to the external opening at locations corresponding to an outer profile of each of the internal tubes such that the external openings are longitudinally-oriented. A plurality of side branch components includes a first side branch component and a second side branch component, each configured to be received in one of the first and second internal tubes and to extend from the external openings in the outer surface of the main body component. The system is configured to be endovascularly implanted in, and to treat the abdominal aorta, the main body component being configured to be anchored in the abdominal aorta at a location that is superior of a location of renal arteries of the patient such that the plurality of side branch components are anchorable in renal arteries of the patient for perfusion thereof, the main body component including at least one of a fenestration feature and a scallop feature configured to align to a superior mesenteric artery of a patient.

The various examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an isometric view of an endovascular treatment system, in accordance with some embodiments.

FIG. 2 is an isometric view of a portion of a main body component of the system of FIG. 1, according to some embodiments.

FIG. 3 is an end view of a main body component of the system of FIG. 1, according to some embodiments.

FIG. 4 is a transverse section of a main body component of the system of FIG. 1, according to some embodiments.

FIG. 5 is an enlarged view of a portion of the transverse section of FIG. 4, according to some embodiments.

FIG. 6 is a view of the main body component and side branch components of the system of FIG. 1 as implanted in an abdominal aorta of a patient with the side branch components received in renal arteries of the patient, according to some embodiments.

FIG. 7 is an isometric view of another endovascular treatment system, in accordance with some embodiments.

FIGS. 8 and 9 are isometric views of a main body component of the system of FIG. 7, according to some embodiments.

FIG. 10 is an end view of the main body component of the system of FIG. 7, according to some embodiments.

FIG. 11 shows a scallop design that may be employed with the main body components of FIGS. 1 and 7, according to some embodiments.

FIG. 12 shows additional views of the main body component of FIG. 1, according to some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### Overview

The various designs addressed in this patent specification relate to an endovascular treatment system 100 including a main body component 102 and a plurality of side branch components 104, including a first side branch component 104a and a second side branch component 104b, although greater or fewer side branch components are contemplated. In various examples, the system 100 is used to treat an aortic aneurysm. In general terms, the system 100 is configured to help direct blood through the aorta while protecting the walls of the aorta from further pressure from the blood flow. In other words, the system 100 acts to carry the blood flow to relieve pressure on damaged, weakened or diseased portions of the vasculature, such as the aorta.

The system 100 is configured to be endovascularly implanted in (e.g., using a catheter delivery system, not shown), and to treat the abdominal aorta. As described in greater detail below (see also, FIG. 6), in various examples, the main body component 102 is configured to be anchored in the abdominal aorta at a location that is superior of, or proximal to, the location the renal arteries (RA) branch from the aorta, and includes branch features for permitting perfusion of those renal arteries. In some examples, the main body component 102 does not extend over the superior mesenteric artery and/or celiac trunk. In other embodiments, the main body component 102 is anchored at a location superior, or proximal, to the superior mesenteric artery and/or celiac trunk and includes features for permitting perfusion for one or both of those aortic branch vessels.

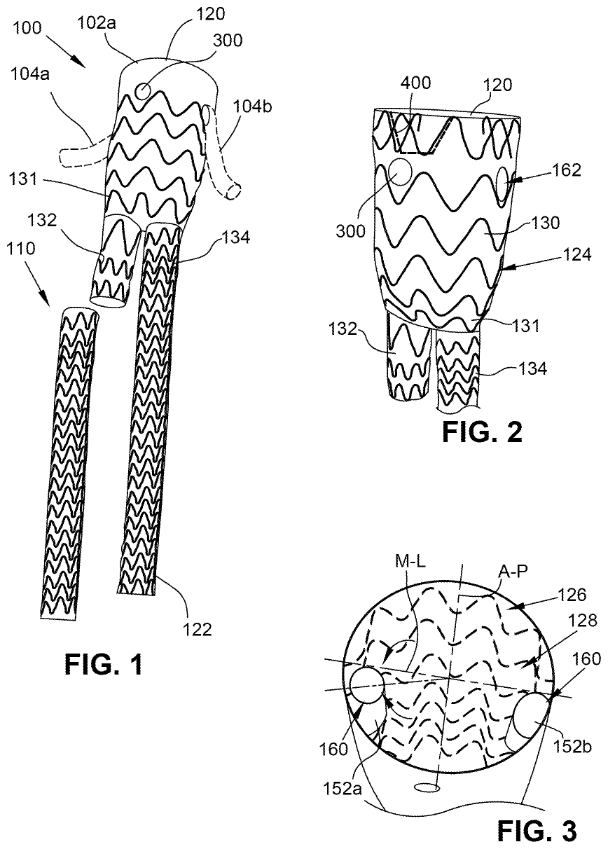

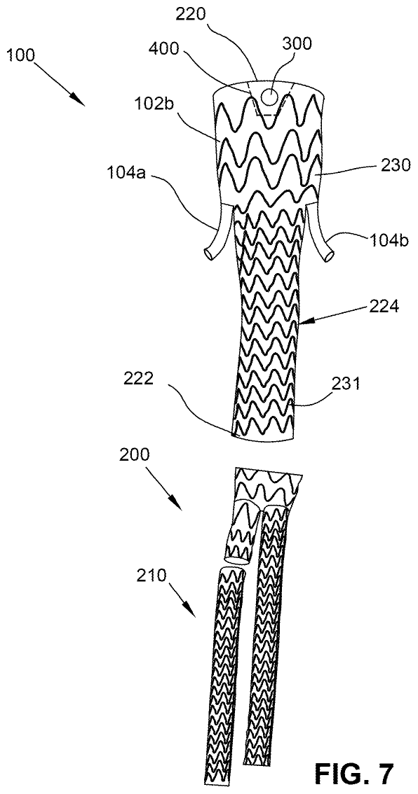

As shown in FIG. 1, for example, the main body component 102 may be a main body component 102a that is terminally bifurcated with a larger proximal inlet and two distal outlets defined by two legs, such as a short leg and a long leg. Or, as shown in FIG. 7, for example, the main body component 102 may be a main body component 102b with a larger, single proximal inlet and a larger, single distal outlet, without defining separate legs or branches at the distal end.

The system 100 may include further stent graft components coupled to the main body component 102 as part of a modular, stent graft solution (e.g., with additional stent graft components secured to the proximal and/or distal end thereof to extend the overall treatment length in a superior direction within the aorta or an inferior direction (e.g., into the common iliac arteries) within the aorta or one or more inferior branches therefrom. For example, as shown in FIG. 1, the system 100 can further include a contralateral leg component 110. The contralateral leg component 110 may be a stent graft configured to be telescopically coupled with the shorter leg of the main body component 102a, also described as a contralateral gate, and to extend into one of the iliac arteries, with the other, longer leg of the main body component 102a (e.g., also described as an ipsilateral leg) extending into the other of the iliac arteries. One example of a suitable contralateral leg component 110 is the contralateral leg component sold as part of the GORE® EXCLUDER® AAA Endoprosthesis system available from W. L. Gore & Associates, Inc.

As another example, as shown in FIG. 7, the system 100 may include a further bifurcated component 200 configured to be telescopically coupled with the distal, or inferior end of the main body component 102b. The bifurcated component 210 may include a shorter leg and a longer leg, with the longer leg (e.g., described as an ipsilateral leg) configured to extend into one iliac artery and the shorter leg configured to couple to a contralateral leg component 210 configured to extend into the other iliac artery. One example of a suitable bifurcated component 200 and contralateral leg component 210 is the GORE® EXCLUDER® Iliac Branch Endoprosthesis system available from W. L. Gore & Associates, Inc.

Each of the side branch components 104 are secured to the main body component 102 at one of a plurality of portal features positioned intermediate the proximal and distal ends of the main body component 102. In various examples, the side branch components 104 serve to perfuse branch vessels that might otherwise be blocked by implantation of the system 100 to treat an aneurysm. For example, the side branch components 104 may be configured to extend from the main body component 102 into the renal arteries to help facilitate ongoing perfusion of the kidneys and/or other organs. As described in greater detail below, in various examples, the main body component 102 (including referenced as 102a, 102b), is configured to be anchored or otherwise extend in a superior, or proximal direction beyond the renal arteries and includes branch features for permitting perfusion thereof.

The main body component 102, side branch components 104, and other components (e.g., leg component 110 and bifurcated component 200) are each formed of a graft component supported by a stent, or frame component. Various materials and methods of manufacture are contemplated for those graft and frame components, including the examples subsequently described.

### Main Body Designs

FIGS. 1 to 6 show various aspects of the main body component 102a according to a first design configuration. As shown, the main body component 102a has a first end 120, also described as a proximal end, a second end 122, also described as a distal end, an outer surface 124, an inner surface 126, and a main lumen 128. The main body component 102 has a proximal portion 130 toward the first end and a distal portion 131 that bifurcates into a first leg 132 that is shorter and a second leg 134 that is longer than the first leg 132. The main lumen bifurcates in the distal portion 131 at the first and second legs 132, 134.

The proximal portion 130 of the main body component 102a is generally configured to be anchored at a position that is superior to a location in the patient anatomy where the renal arteries branch from the aorta. The proximal portion 130 may include tissue anchors for gripping vascular tissue of the aorta to reduce the risk of migration from the anchor position. As shown, the proximal portion 130 of the main body component 102a includes a plurality of internal tubes 152, including a first internal tube 152a and a second internal tube 152b that define portal features into which the branch components 104 may be secured. Each of the internal tubes 152 has an inlet or origin 160 located within the main lumen 128 and terminates at an external opening 162 in the outer surface 124 of the main body component 102a. The external opening 162 may be framed, or bounded by one or more of the stent or frame elements (e.g., one of the undulations, or apices may help define an inferior edge and/or the sides of the external opening 162. As shown, the external opening 162 is flush with the outer surface 124. And, as shown, the origin 160 is oriented toward the first end 120 and is generally longitudinally aligned with the main lumen 128. In turn, the external opening 162 extends substantially transverse, or perpendicular to the main lumen 128. The internal tubes 150 generally extend in a longitudinal direction of the main lumen 128.

The internal tubes 152 generally define portals, or gates, into which side branch endoprostheses may be affixed to direct flow from the main body component 102a to one or more branch vessels, such as the renal arteries. Although two internal tubes are shown, any number are contemplated and the current embodiment shown is provided by way of example.

The internal tubes 152 (also described as branch tubes) are optionally made by adding additional graft material that is formed into a tube and coupled (e.g., sewn and/or adhered) to the internal side of the main body (usually the graft component). Each of the internal tubes 152 are optionally supported by a framework, or stent component. The internal tubes 152 are sized to engage and secure one of the side branch components 104 (represented generally by broken lines in FIG. 1) such that they may protrude from the external openings 162 into a side branch vessel.

As shown in FIG. 3, the internal tubes 152 are positioned located at a relative angular position, or clocked at a location around the inner circumference of the main body component 102a. FIG. 3 has a medial-lateral M-L and anterior-posterior A-P coordinate system indicated passing through the longitudinal centerline of the main body component 102a. The first and second internal tubes 152a, b may be clocked to the medial-lateral axis or offset therefrom. In such an example, the first and second internal tubes 152a, b are offset by approximately 180 degrees from one another+/−20 degrees. In some examples the first and second internal tubes 152a, b are each anteriorly offset from the medial-lateral axis, for example by 5 degrees, 10 degrees, 15 degrees, 20 degrees, or some other offset as desired. As shown, the origins 160 are longitudinally aligned, or substantially longitudinally aligned, although they may be offset from one another as desired. In some examples, the origins 160 are inset from the first end 120 of the main body component 102a from 5 mm to 10 mm, for example, although a variety of offsets are contemplated. In various examples, the external openings 162 are offset from the first end 120 of the main body 102a by no more than 40 mm, for example, although a variety of offsets are contemplated.

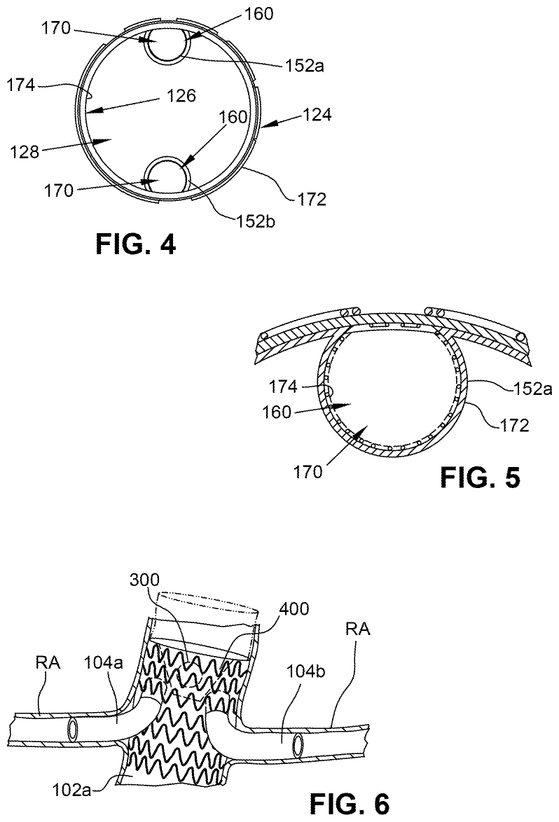

FIGS. 4 and 5 are transverse sections of the proximal portion 130 at a location between the origins 160 and external openings of the internal tubes 152. As shown, the internal tubes 152 define secondary lumens 170 between innermost graft layer(s) 172 and outermost graft layer(s) 174. The internal tubes 152 include a secondary framework, or stent 176. The internal tubes 152 may include additional features such as radiopaque markers.

Suitable examples of internal tube constructs for the internal tubes 152 may also be found in U.S. Pat. No. 10,653,540 to Hagaman et al. and U.S. Pat. No. 6,645,242 to Quinn.

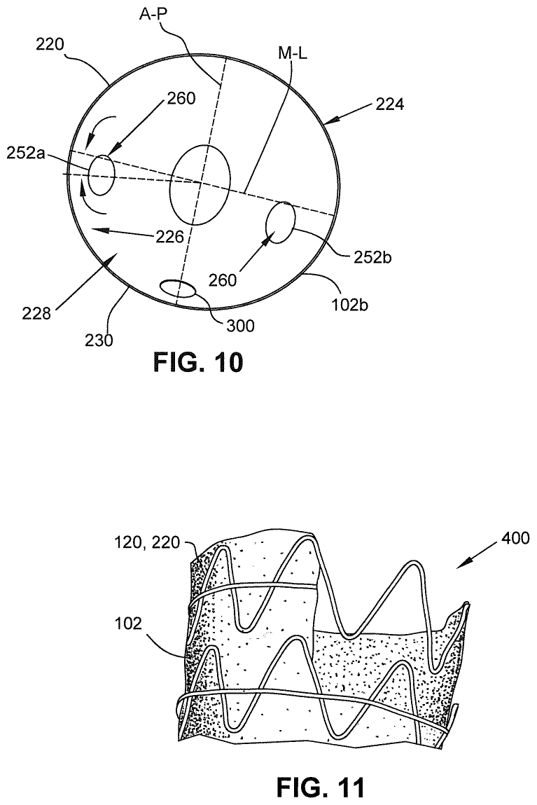

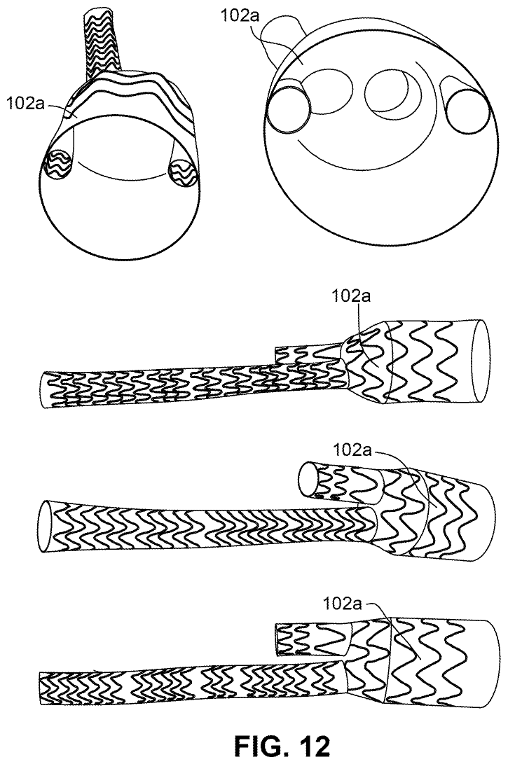

FIGS. 7 to 10 show various aspects of the main body component 102b according to a second design configuration. As shown, the main body component 102b has a first end 220, also described as a proximal end, a second end 222, also described as a distal end, an outer surface 224, an inner surface 226, and a main lumen 228 (FIGS. 9 and 10). The main body component 102b has a proximal portion 230 toward the first end and a distal portion 231 that tapers to a narrower diameter from the proximal portion 230.

The proximal portion 230 of the main body component 102b is generally configured to be anchored at a position that is superior to a location in the patient anatomy where the renal arteries branch from the aorta. The proximal portion 230 may include tissue anchors for gripping vascular tissue of the aorta to reduce the risk of migration from the anchor position. As shown in FIG. 10, the proximal portion 230 of the main body component 102b includes a plurality of internal tubes 252, including a first internal tube 252a and a second internal tube 252b that define portal features into which the branch components 104 may be secured. As shown in FIG. 10, each of the internal tubes 252 has an inlet or origin 260 located within the main lumen 228 and terminates at an external opening 262 (FIG. 9) in the outer surface 224 of the main body component 102b. The origin 260 is oriented toward the first end 220 and is generally longitudinally aligned with the main lumen 228. The external opening 262 extends substantially parallel, or aligned to the main lumen 228. As indicated, the internal tubes 250 generally extend in a longitudinal direction of the main lumen 228.

The internal tubes 252 generally define portals, or gates, into which side branch endoprostheses may be affixed to direct flow from the main body component 102b to one or more branch vessels, such as the renal arteries. Although two internal tubes are shown, any number are contemplated and the current embodiment shown is provided by way example. As shown, the outer surface 224 is enlarged, raised, or otherwise projects radially outward to define the outer profile of the internal tubes 252 toward the external opening 262.

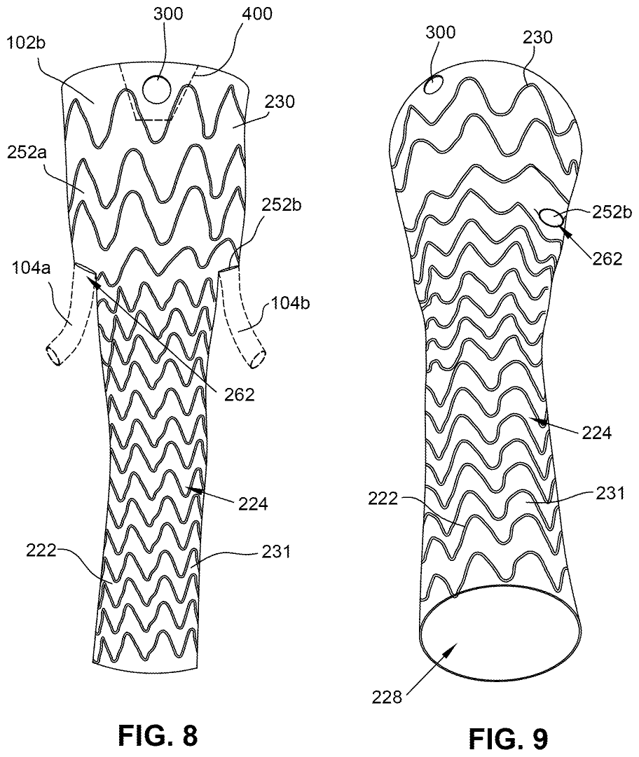

Similarly to the internal tubes 152, the internal tubes 252 (also described as branch tubes) are optionally made by adding additional graft material that is formed into a tube and coupled (e.g., sewn and/or adhered) to the internal side of the main body (usually the graft component). Each of the internal tubes 252 is optionally supported by a framework, or stent component. The internal tubes 252 are sized to engage and secure one of the side branch components 104 (represented generally by broken lines in FIGS. 7 and 8) such that they may protrude from the external openings 262 into a side branch vessel.

As indicated in FIG. 10, the internal tubes 252 are positioned located at a relative angular position, or clocked at a location around the inner circumference of the main body component 102b. FIG. 10 has a medial-lateral M-L and anterior-posterior A-P coordinate system indicated passing through the longitudinal centerline of the main body component 102b. The first and second internal tubes 252a, b may be clocked to the medial-lateral axis or offset therefrom. In such an example, the first and second internal tubes 252a, b are offset by approximately 180 degrees from one another+/−20 degrees. In some examples, the first and second internal tubes 252a, b are each anteriorly offset from the medial-lateral axis, for example by 5 degrees, 10 degrees, 15 degrees, 20 degrees, or some other offset as desired. As shown, the origins 260 are longitudinally aligned, or substantially longitudinally aligned, although they may be offset from one another as desired. In some examples, the origins 260 are inset from the first end 220 of the main body component 102b from 5 mm to 10 mm, for example, although a variety of offsets are contemplated. In various examples, the external openings 262 are offset from the first end 220 of the main body 102b by no more than 40 mm, for example, although a variety of offsets are contemplated.

FIGS. 4 and 5 are also generally representative transverse sections of the proximal portion 230 at a location between the origins 260 and external openings of the internal tubes 252. Suitable examples of internal tube constructs for the internal tubes 252 may also be found in U.S. Pat. No. 10,653,540 to Hagaman et al. and U.S. Pat. No. 6,645,242 to Quinn.

### Fenestration Designs

As represented by broken lines in various figures, the main body component 102 (including example main body components 102a, 102b) may include one or more fenestration features 300. The one or more fenestration features 300 may be positioned between two or more stent, or framework rings, or turns. The one or more fenestration features 300 may be pre-fenestrated, or fenestratable. In other words, the graft component of the main body component 102 may be removed with a void (pre-fenestrated) or the graft component may be configured to be removable to form a void (e.g., ablated, cut, punctured or otherwise opened to form the one or more fenestration features 300). The one or more fenestration features 300 may include a frame member support (e.g., a ring of material similar to a stent member) and may be radiopaque as desired. In other examples, the one or more fenestration features 300 are unsupported by a frame member, and may be radiopaque (or have edges delineated by radiopaque material) as desired. The one or more fenestration features 300 may be arranged to align to a side branch vessel, such as the superior mesenteric artery to facilitate perfusion thereof. Additionally or alternatively, the one or more fenestration features 300 may receive a side branch component or other feature.

### Scallop Designs

In some embodiments, the main body component 102 may include one or more scallop features 400 at the first end (e.g., first end 120, 220), as designated in various drawings by a broken line boundary. The scallop feature 400 may be implemented to facilitate placement of the main body component 102 in a lumen including a side branch vessel that does not need a prosthetic side branch deployed. For example, when the main body component 102 is positioned in the abdominal aorta and the superior mesenteric artery does not need a side branch 14 deployed therein, the scallop feature 400 may be positioned over the entrance into the superior mesenteric artery without blocking or restricting blood perfusion therethrough. The scallop feature 400 may include various shapes including straight edge profiles, curved profiles, and combinations thereof. One, generally trapezoidal shaped void in the graft component is shown in broken lines by way of example. As shown, the stent component optionally extends over the void, although embodiments where the stent component is also removed, or shaped to the scallop feature 400, for example, are also contemplated. Similarly to the fenestration feature(s), the scallop feature 400 may include radiopaque material to designate its edges or boundaries, for example.

FIG. 11 shows one example of a design for scallop feature 400. As shown in FIG. 11, the scallop feature 400 is located at the proximal end 120,200 and is defined by a void in the graft component forming the main body 102. As shown, the stent component extends over the scallop feature 400. In this manner, support and anchoring functionality may still be provided to the main body 102 by the stent component, while still allowing blood flow to pass through the scallop feature 400 for perfusion purposes (e.g., to perfuse a superior mesenteric artery of a patient).

### Branch Graft Designs

The side branch components 104 may take a variety of forms, including self-expanding and balloon expandable stent graft configurations. Generally, the side branch components 104 are sized and shaped to be received in one of the internal tubes 150 and extend into a desired side branch vessel, such as the renal arteries. One example of a suitable side branch component corresponds to the GORE® VIABAHN® VBX Balloon Expandable Endoprosthesis available from W. L. Gore & Associates, Inc. Another suitable example, corresponds to the GORE® VIABAHN® Endoprosthesis (self-expanding) available from W. L. Gore & Associates, Inc.

### Materials

The materials used for the graft components associated with the main body and branch components can include any material which is suitable for use as a graft in the chosen body lumen. The graft components for the main body and branch(es) can be composed of the same or different materials. The graft components can comprise multiple layers of material that can be the same material or different materials. Although the graft components can have several layers of material, the graft components may have a layer that is formed into a tube (innermost tube) and an outermost layer that is formed into a tube (outermost tube).

Many graft materials are known, particularly known are those that can be used as vascular graft materials. The graft materials can be extruded, coated or formed from wrapped films, or a combination thereof.

Polymers, biodegradable and natural materials can be used for specific applications. Biocompatible materials in particular are contemplated for the various graft components associated with the main body and branch components described herein. In certain instances, the graft components may include a fluoropolymer, such as a polytetrafluoroethylene (PTFE) polymer or an expanded polytetrafluoroethylene (ePTFE) polymer. In some instances, the graft components may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

Biocompatible materials may be used for the various frame components, or stent components, associated with the main body and branch components described herein. For example, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

### Notice

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An endovascular treatment system comprising:
a main body component having a first end, a second end, and an inner lumen, the main body including a first internal tube and a second internal tube located within the inner lumen, each of the first and second internal tubes having an origin oriented toward the first end of the main body component and an external opening in an outer surface of the main body component; and
a plurality of side branch components, including a first side branch component and a second side branch component, each configured to be received in one of the first and second internal tubes and to extend from the external openings in the outer surface of the main body component,
wherein the system is configured to be endovascularly implanted in, and to treat the abdominal aorta, the main body component being configured to be anchored in the abdominal aorta at a location that is superior of a location of renal arteries of the patient such that the plurality of side branch components are anchorable in renal arteries of the patient for perfusion thereof, the main body component including at least one of a fenestration feature and a scallop feature configured to align to a superior mesenteric artery of a patient.

2. The system of claim 1, wherein the first and second internal tubes are circumferentially offset by approximately 180 degrees.

3. The system of claim 1, wherein the first and second internal tubes are circumferentially offset by approximately 160 degrees.

4. The system of claim 1, wherein the first and second internal tubes are circumferentially offset by approximately 140 degrees.

5. The system of claim 1, wherein the main body component has a larger, proximal inlet and two smaller, distal outlets defined by two legs.

6. The system of claim 5, wherein the two legs include a short leg and a long leg.

7. The system of claim 1, wherein the main body component has a larger, single proximal inlet and a larger, single distal outlet.

8. The system of claim 1, wherein the main body has an outer surface that projects radially outward proximate the external opening at locations corresponding to an outer profile of each of the internal tubes such that the external openings are longitudinally-oriented.

9. The system of claim 1, wherein the origins of each of the first and second internal tubes are oriented toward the first end of the main body and are longitudinally aligned with the inner lumen of the main body, and further wherein the external openings of each of the first and second internal tubes are longitudinally aligned to the inner lumen.

10. The system of claim 1, wherein the main body has an outer surface and further wherein the external openings of the first and second internal tubes are flush with the outer surface of the main body such that the external openings are radially-oriented.

11. The system of claim 1, wherein the origins of each of the first and second internal tubes are oriented toward the first end of the main body and are longitudinally aligned with the inner lumen of the main body, and further wherein the external openings of each of the first and second internal tubes extend transverse to the inner lumen.

12. The system of a claim 1, wherein the origin of at least one of the first and second internal tubes are inset from the first end of the main body by an inset distance.

13. The system of claim 1, wherein the inset distance is from 5 mm to 10 mm.

14. The system of claim 1, wherein the external opening of at least one of the first and second internal tubes are offset from the first end of the main body by an offset distance of no more than 40 mm.

15. A method of delivering an endoprosthesis to a treatment site in vasculature of a patient, the method comprising:
delivering a main body component having a first end, a second end, and an inner lumen to a location in the abdominal aorta such that the first end of the main body is positioned at a location superior to renal arteries of the patient, wherein the main body including a first internal tube and a second internal tube located within the inner lumen, each of the first and second internal tubes having an origin oriented toward the first end of the main body component and an external opening in an outer surface of the main body component;
aligning at least one of a fenestration feature and a scallop feature of the main body component to a superior mesenteric artery of the patient;
delivering a plurality of side branch components, including a first side branch component in the first internal tube and a second side branch component in the second internal tubes such that the first and second side branch components extends from respective ones of the external openings in the outer surface of the main body component; and
anchoring the first end of the main body component superior to the renal arteries and anchoring the first and second branch components in respective ones of the renal arteries for perfusion thereof.

16. The method of claim 15, wherein the first and second internal tubes of the main body component are circumferentially offset by approximately 180 degrees.

17. The method of claim 15, wherein the first and second internal tubes are circumferentially offset by approximately 160 degrees.

18. The method of claim 15, wherein the first and second internal tubes are circumferentially offset by approximately 140 degrees.

19. The method of claim 15, wherein the main body component has a larger, proximal inlet and two smaller, distal outlets defined by two legs.

20. An endovascular treatment system comprising:
a main body component having a first end, a second end, and an inner lumen, the main body including a first internal tube and a second internal tube located within the inner lumen, each of the first and second internal tubes having an origin oriented toward the first end of the main body component and an external opening in an outer surface of the main body component, the first and second internal tubes being circumferentially offset from approximately 140 degrees to approximately 180 degrees from one another, the main body having an outer surface that projects radially outward proximate the external opening at locations corresponding to an outer profile of each of the internal tubes such that the external openings are longitudinally-oriented; and
a plurality of side branch components, including a first side branch component and a second side branch component, each configured to be received in one of the first and second internal tubes and to extend from the external openings in the outer surface of the main body component,
wherein the system is configured to be endovascularly implanted in, and to treat the abdominal aorta, the main body component being configured to be anchored in the abdominal aorta at a location that is superior of a location of renal arteries of the patient such that the plurality of side branch components are anchorable in renal arteries of the patient for perfusion thereof, the main body component including at least one of a fenestration feature and a scallop feature configured to align to a superior mesenteric artery of a patient.
